(19) United States
(12) Patent Application Publication
(10) Pub. No.: US 2006/0121019 A1
(43) Pub. Date: Jun. 8, 2006

(54) Compositions for treating cystic fibrosis

(76) Inventors: John A. Budny, Westlake Village, CA (US); Matthew J. Budny, Westlake Village, CA (US)

Correspondence Address: COLIN P ABRAHAMS, 5850 CANOGA AVENUE, SUITE 400, WOODLAND HILLS, CA, 91367, US

(21) Appl. No.: 11/242,447

(22) Filed: Oct. 3, 2005

Related U.S. Application Data
(63) Continuation of application No. 09/875,997, filed on Jun. 6, 2001, now abandoned.
Application No. 09/875,997 is a continuation-in-part of application No. 09/587,818, filed on Jun. 6, 2000, now Pat. No. 6,830,745.
Application No. 09/587,818 is a continuation-in-part of application No. 09/249,674, filed on Feb. 12, 1999, now Pat. No. 6,159,447.
Application No. 09/249,674 is a continuation-in-part of application No. 08/951,393, filed on Oct. 16, 1997, now Pat. No. 5,871,714.

(51) Int. Cl. A61K 38/47

(52) U.S. Cl. 424/94.61

(57) ABSTRACT

A composition for degrading biofilm structure associated with cystic fibrosis and the debris associated therewith comprises an enzyme selected for its ability to dismantle the biofilm structure, and an anchor molecule coupled to an enzyme to form an enzyme-anchor complex. The anchor molecule is selected for its ability to attach to a surface on or proximal the biofilm structure. The attachment to the surface permits prolonged retention time of the enzyme-anchor complex where the biofilm structure and associated debris are present.

### CROSS REFERENCE TO RELATED APPLICATIONS

This application is continuation of application Ser. No. 09/875,997 filed Jun. 6, 2001, a continuation-in-part of U.S. application Ser. No. 09/587,818 filed Jun. 06, 2000, which is a continuation-in-part of U.S. application Ser. No. 09/249,674 filed Feb. 12, 1999 (issued as U.S. Pat. No. 6,159,447 on Dec. 12, 2000), which is a continuation-in-part of U.S. application Ser. No. 08/951,393 filed Oct. 16, 1997 (issued as U.S. Pat. No. 5,871,714 on Feb. 16, 1999), both of which are incorporated herein by reference.

### FIELD AND BACKGROUND OF THE INVENTION

Cystic fibrosis, a genetically inherited disease, is caused by the mutation of a gene that produces an electrolyte transfer protein. The consequence of the mutation affects a multitude of organ systems. However, the tissues that are most directly affected are those that secrete mucus or have mucus membranes. Serious adverse consequences occur with tissues that are associated with the respiratory system i.e. lungs and airway passage tissues. In response to the genetic defect, the host produces secretions to counteract the ionic imbalance. These secretions in response to the disease allow opportunistic infections to develop which causes additional fluid and mucus to infiltrate the respiratory system from the host. The additional fluid from the host in response to the infection is under the influence of the immune system. Finally, the infectious bacteria and the associated bacterial biofilm add additional mucus and fluid at the site. Furthermore, the principal bacterial pathogen associated with the opportunistic infection is Pseudomonas aeruginosa, which often mutates to a mucoid form that is a prolific producer of alginate biofilm, which further exacerbates the disease condition. The result is an accumulation of copious amount of mucus, fluid and biofilm material which affects not only the respiratory system, but the entire host.

The current treatment of cystic fibrosis involves a dual approach to: 1) promote and facilitate the removal of mucus and secretions from the respiratory tract and; 2) control the infection that is associated with the disease. The infection, with the bacteria's production of a biofilm, obstructs the host's defenses, shields the bacteria from the killing action of antibiotics and increases the viscosity of the mucus, making it increasingly difficult for the patient to expectorate the interfering mucus.

Currently, there are limitations in achieving these objectives. Existing pharmacological agents that assist in removing mucus from the respiratory tract are not completely effective. In addition, since chronic use of antibiotics are necessary to treat the on-going infection, resistance develops, making effective antibiotic treatment increasingly difficult. Consequently, a therapeutic strategy, which is focused on both the microbial biofilm and the mucus, is an attractive treatment regimen for the disease.

Attacking and dismantling a biofilm structure is described by Budny (U.S. Pat. No. 5,871,714) and Budny, et al. (U.S. Pat. No. 6,159,447). They teach a composition for controlling bacterial growth and colonization by using an enzyme-anchor complex to dismantle bacterial biofilms. The result is reduced bacterial colony size and a reduction in the total number of bacteria.

Based on work done in the 1950s and 1960s and summarized by Skak et al. [Proc. Natl. Acad. Sci. 87, 9188-9192(1990)], the viscosity of the sputum from cystic fibrosis patients was reduced when the sputum was treated with the enzyme DNase. Consequently, a less viscous, easier flowing mucus is more readily expectorated.

Infections in cystic fibrosis patients of the lower respiratory tract pose a unique requirement for treatment. These infections represent a long-term, continual infection that is confounded by periodic acute infection episodes in the upper respiratory tract. Aerosolized antibiotics have been used and in certain cases, surgical interventions have been used in addition to antibiotics.

### SUMMARY OF THE INVENTION

According to one aspect of the invention, there is provided a composition for degrading biofilm structure associated with cystic fibrosis and the debris associated therewith, the composition comprising: an enzyme selected for its ability to dismantle the biofilm structure; an anchor molecule coupled to an enzyme to form an enzyme-anchor complex, the anchor molecule being selected for its ability to attach to a surface on or proximal the biofilm structure; wherein the attachment to the surface permits prolonged retention time of the enzyme-anchor complex where the biofilm structure and associated debris are present.

Bacteria, living in colonies, produce a biofilm that not only provides an environment for the bacteria to have close proximity to one another, but also protects the community from external challenges. The challenges originate from the host's immune system and from antibiotics, which are administered to treat the infection. Consequently, dismantling the biofilm makes the colonies, as well as the individual bacteria, more susceptible to the host's defenses and more vulnerable to the action of antibiotics.

One aspect of the invention consists of one or more enzyme(s) whose specificity includes its (their) ability to degrade the polysaccharide backbone of the biofilm structure produced by Pseudomonas aeruginosa that occurs in the respiratory tract of cystic fibrosis patients. While these polysaccharide-degrading enzymes are hydrolytic, they are found in three major classifications: Carboxylic Ester Hydrolases (EC 3.1.1.-), Glycosidases (EC 3.2.-.-) and Lyases Acting on Polysaccharides (EC 4.2.2.-). Examples of these enzymes include, but are not limited to, acetyl esterases, polyguluronate hydrolase, polymannuronate hydrolase, alginate lyase and alginate depolymerase.

Attached to the enzyme(s), either through chemical synthetic procedures or recombinant technology, are one or more moieties that have the capability of binding, either reversibly (non-covalently) or irreversibly (covalent bonded) to a surface near the biofilm or the biofilm itself. Collectively, these moieties are called anchors. The moieties, selected to serve as anchors, can be agents or molecular species known to have an affinity for the biofilm, the surfaces near the biofilm or known binding domains. Examples of these types of anchors are listed below. The listing is not intended to be a complete list; rather, the listed examples serve to illustrate the entire class. Finally, the search for anchors can be accomplished with High Throughput Screening (HTS) of a biofilm of either known or unknown composition with various molecular entities using a suitable assay to determine which materials have an affinity for the biofilm or surrounding surfaces.

These two properties: 1. an enzyme; and 2. a binding component that is connected to the enzyme, are directed at the degradation of the biofilm backbone structure.

Moieties with a Known Affinity for Biofilms
Concanavalin A
Wheat Germ Agglutinin
Other Lectins
Heparin Binding Domains
Elastase
Amylose Binding Protein
Ricinus communis agglutinin I (RCA I); Dilichos biflorus agglutinin (DBA); Ulex europaeus agglutinin I (UEA I).

Binding Domains from the Following Enzymes
Dextransucrase
Starch-synthesizing enzymes
Cellulose-synthesizing enzymes
Chitin-synthesizing enzymes
Glycogen-synthesizing enzymes
Pectate synthetase
Glycosyl transferase-binding domains (glucan-, mutan-, levan-, Polygalactosyl-synthesizing enzymes et al.

Certain agents have been described (U.S. Pat. Nos. 3,309,274, 3,624,219, 4,064,229 and 4,431,628) as indicators or disclosing agents for oral bacterial biofilms. In effect, these agents bind to the biofilm where they can be visualized either by the naked eye or with the aid of a light source with a wavelength that shows the agent's color. The purpose of these agents as described in the cited patents is to show location of the biofilm structure.

Since biofilm disclosing agents have the required property to bind to a biofilm, they make exceptionally good anchors in the enzyme-anchor complexes. Consequently, any molecular entity whose purpose is to serve as a biofilm disclosing agent can also be used as an anchor for the anchor enzyme complex to retain enzymes at or near a biofilm. The following is a list of examples of biofilm disclosing agents, which are examples of molecules that can serve as anchors. This list is only a selected list of examples and it is not intended to exclude other disclosing agents.

Examples of Biofilm Disclosinc Agents
FD&C Red #3 (erythrosin)
Amaranth (Brilliant Blue)
Synthetic fluorescent dyes
D&C Green #8
D&C Red #s 19, 22 and 28
D&C Yellow #s 7 and 8
Natural fluorescent dyes
Chlorophyll dye
Carotene
FD&C Blue #1
FD&C Green #3
Hercules Green Shade 3
Merbromin
Betacyanines
Betamine
Betanin
Betaxanthines
Vulgaxathin
Ruthenium Red.

Another and second aspect of the invention consists of two or more hydrolytic enzymes. One enzyme has the specificity to degrade the biofilm's polysaccharide backbone structure of a biofilm; at least one other enzyme is hydrolytic in nature, having the capability to degrade extraneous byproducts of both the biofilm and host-originated materials. These extraneous materials, host-bacteria- and biofilm-derived byproducts include immuno-chemicals such as proteins, polypeptides, glycoproteins, nucleic acids, lipids, lipid complexes of sugars and proteins (lipopolysaccharides and lipoproteins).

Blends and combinations of enzymes have been used for industrial processing applications and that multiple enzymes, used together, can remove biofilms (Johansen, C., Falholt, P. and Gram, L. “Enzymatic Removal and Disinfections of Bacterial Biofilms.” Applied and Environmental Microbiology, Vol. 93, No. 9, September 1997, p. 3724-3728). As an illustrative example, alginate lyase, pectinase, arabinase, cellulase, hemicullulase, β-glucanase and xylanase each connected to elastase, with the elastase serving as an anchor to the biofilms can be used to remove alginate biofilms. Alginate biofilms ordinarily produced by Pseudomonas aeruginosa and Pseudomonas fluorescens; however, the enzyme-anchor complex described herein, will effectively remove alginate-based biofilms produced by any bacterial or fungal species, whether they act alone or in combination with one another to create the biofilm.

The enzymes capable of degrading proteins and polypeptides are found in classification EC 3.4.-.-. These proteinases include proteolytic enzymes, endopeptidases, peptidyl-peptide hydrolases, serine proteinases, acid proteinases and SH-proteinases. In a universal sense, all of the protein and peptide hydrolysis enzymes cleave the amide linkage between adjacent amino acids in either a polypeptide or protein. Specific examples would include, but not be limited to, peptidases, carboxypeptidase, particle-bound amino peptidase (EC 3.4.11.2), chymotrypsin, trypsin, cathepsin, thrombin, prothrombinase, plasmin, elastase, subtilsin, papain, ficin, asclepain, pepsin, chymosin, collagenase and those enzymes with EC 3.4.99.-, which possess proteinase activity of unknown mechanisms.

Many of the enzymes that hydrolyze glycoproteins (proteoglycans) and nucleic acids have not been specifically isolated and characterized, with known mechanisms of actions. Those proteinases, peptidyl-hydrolyases and nucleic acid hydrolases where the mechanism is not known are initially classified in either EC 3.-.- as hydrolases, most likely falling into EC 3.1.-, EC 3.2.-, EC 3.4.-, and EC 4.2.2.- (Lyases Acting on Polysaccharides). Some currently identified enzymes that hydrolyze glycoproteins include:
Peptidoglycan endopeptidase(hydrolase) (EC 3.4.99.17)
Heparin lyase(EC 4.2.2.7)
Heparatinase
Chitodextrinase (EC 3.2.1.14)
Chondroitin lyase (EC 4.2.2.4; EC 4.2.2.5)
Muramindase (EC 3.2.1.17)
N-Acetylmuramidase
Sialidase/Neuraminidase (EC 3.2.1.18)
β-N-Acetylhexosaminidase (EC 3.2.1.52)
α-N-Acetylhexosaminidase
β-N-Acetylglucosaminidase (EC 3.2.1.30)
Hyaluronoglucosidase (EC 3.2.1.35)
Hyaluronoglucuronidase (EC 3.2.1.36)
β-N-Acetylgalactosaminidase (EC 3.2.1.53)
β-Aspartylacetylglucosaminidase (EC 3.2.2.1) et al.

Some identified hydrolases acting on nucleic acid material from the general class of EC 3.1.7.- to EC 3.1.31.- include, but are not limited to, the following:
exo-deoxyribonuclease I (EC 3.1.11.1)
exo-deoxyribonuclease Iii (EC 3.1.11.2)
exo-deoxyribonuclease (Lambda-induced) (EC 3.1.11.3)
exo-deoxyribonuclease (Phage Sp3-induced) (EC 3.1.11.4)
exo-deoxyribonuclease V (EC 3.1.11.5)
exo-deoxyribonuclease Vii (EC 3.1.11.6)
exo-ribonuclease (EC 3.1.13.-)
exo-ribonuclease. (EC 3.1.14.-)
Venom exo-nuclease (non-specific) (EC 3.1.16.1)
Spleen exo-nuclease (non-specific) (EC 3.1.16.1)
endo-deoxyribonuclease I (EC 3.1.21.1)
endo-deoxyribonuclease Iv (EC 3.1.21.2)
endo-deoxyribonuclease (Type I specific) (EC 3.1.21.3)
endo-deoxyribonuclease (Type Ii specific) (EC 3.1.21.4)
endo-deoxyribonuclease (Type Iii specific) (EC 3.1.21.5)
endo-deoxyribonuclease (C-C preferred) (EC 3.1.21.6)
Deoxyribonuclease Ii (EC 3.1.22.1)
Aspergillus Deoxyribonuclease K1 (EC 3.1.22.2)
Deoxyribonuclease V (EC 3.1.22.3)
endo-ribonuclease (Crossover) (EC 3.1.22.4)
Deoxyribonuclease X (EC3.1.22.5)
Deoxyribonuclease (pyrimidine dimmer) (EC 3.1.25.1)
Deoxyribonuclease (EC3.1.25.2)
endo-ribonuclease (EC 3.1.26.-)
endo-ribonuclease (EC 3.1.27.-)
Aspergillus nuclease (EC 3.1.30.1)
Serratia marcescens nuclease (EC 3.1.30.2)
Micrococcal nuclease (EC 3.1.31.1)

Attached to the enzymes, either individually or collectively as a single unit through chemical synthetic procedures or recombinant technology, are one or more moieties that have the capability of binding either reversibly (non-covalently) or irreversibly (covalent bonded) to a surface near the biofilm or the biofilm itself. This aspect is directed at the degradation and removal of the biofilm backbone structure along with any other materials that may be associated with the backbone, which collectively constitute the entire biofilm. The potential anchors were described under the first aspect of the invention, above.

Still another aspect of the invention consists of two or more enzymes, wherein at least one enzyme has the capability of degrading a biofilm structure produced by a bacterial strain, or a mixed combination of various strains, and the other enzymes(s) has (have) the capability of acting directly upon the bacteria, causing lysis of the bacterial cell wall. One or more moieties are attached to the enzymes, forming either a single unit or multiple units. The moieties are attached to the enzymes either through chemical synthetic procedures or recombinant technology to give the enzyme moiety the capability of binding either reversibly (non-covalently) or irreversibly (covalent bonded) to a surface near the biofilm or the biofilm itself. The purpose of this multi-enzyme system is directed at the degradation and removal of the biofilm with the contemporaneous bactericidal consequences for bacteria that were embedded in the biofilm's structure have become exposed due to the action of the biofilm-degrading enzyme(s).

Lysozyme has long been known to have bactericidal activity by destroying the bacterial cell wall, freeing cell wall components, which leads to cell lysis. Anchored lysozyme, along with anchored polysaccharide-degrading enzyme(s), can be used in concert to remove the polysaccharide backbone of a biofilm and then lyse the resident bacteria in a stepwise fashion. In a specific example of the removal of oral biofilms, lysozyme can be connected to amylase binding protein or the glucan-binding domain, either by coupling the lysozyme to the selected anchor or through a recombinant synthesis. The consequence of this combination is that the polysaccharide backbone is removed and the embedded bacteria are killed through cell lysis at the same time.

Lysozyme can be used in the treatment and removal of other. biofilms along with the resident bacteria that may exist outside of the oral cavity. For biofilms produced by Pseudomonas aeruginosa and Pseudomonas fluorescens, lysozyme can be anchored with elastase and used in conjunction with any one of the following biofilm-degrading enzymes: alginate lyase, pectinase, arabinase, cellulase, hemicullulase, β-glucanase and/or xylanase, each connected to elastase or some other suitable anchor.

This multi-enzyme, dual functionality for treating and eliminating biofilms can be used for any microorganism that produces a biofilm e.g., fungi and yeasts.

Examples of enzymes that have the capability to kill bacteria are as follows: Lysozyme (EC 3.2.1.17); Mucinase (EC 3.2.1.35); Neuraminidase (EC 3.2.1.18); Keratanase (EC 3.2.1.103); Capsular polysaccharide galactohydrolase (EC 3.2.1.87); Glycoside hydrolase (EC 3.2.1.-); Chondroitin ABC lyase (EC 4.2.2.4); Heparatinase; Heparin lyase (EC 4.2.2.4); Glycosaminoglycan lyases (EC 4.2.2.-); Pectate lyase (EC 4.2.2.2); Peptidoglycan hydrolase (Lysostaphin) (EC 3.4.99.17); any bacteriophage polysaccharide depolymerase; Holin enzymes.

Many bacteriophage enzymes require specific proteins that assist in the penetration of the lytic enzyme into the bacterial well wall. These proteins, called holins, may be associated with the genes that encode the lytic enzymes. Holins are believed to assist the lytic enzymes to gain access to the components of the bacterial cell wall that serve as a substrate for the enzyme. These holing proteins may be enzymes themselves.

### BRIEF DESCRIPTION OF THE DRAWINGS

FIG. 1 is a schematic view of a biofilm from a distance;

FIG. 2 is a schematic view showing the elements of a single layer within a biofilm structure;

FIG. 3 is a schematic view of a magnified section of a single biofilm layer; and

FIG. 4 is a diagram of a Robbins-type flow cell to measure biofilm dynamics under various flow conditions and components that may be added to the flowing fluid.

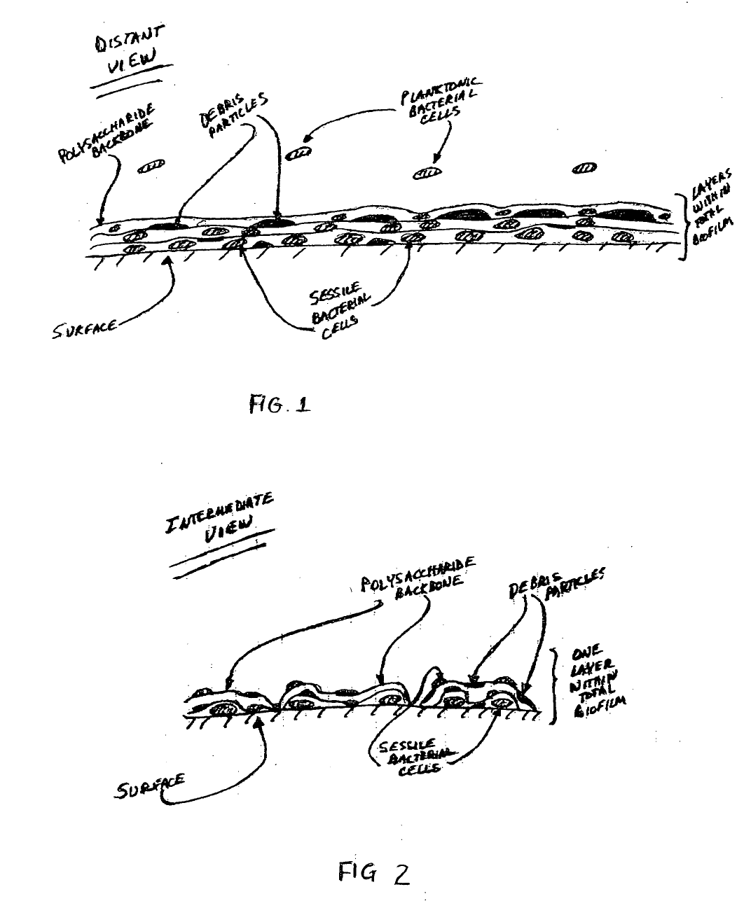

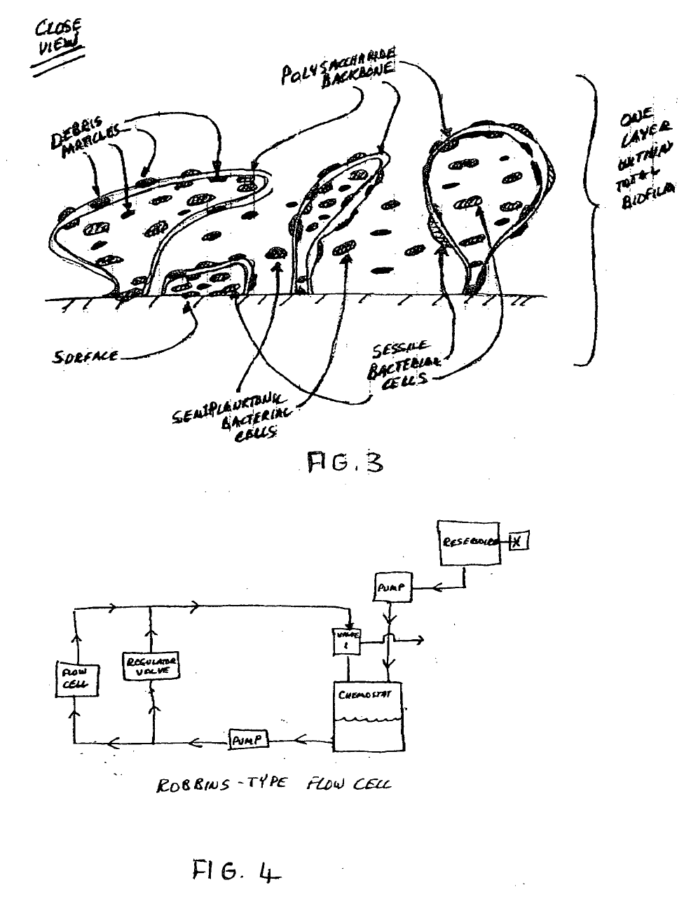

### DETAILED DESCRIPTION OF THE INVENTION

The invention will be described using Pseudomonas aeruginosa by way of example Pseudomonas aeruginosa was selected as an example because it is associated with the genetic-based disease of cystic fibrosis. However, the principles described in this invention apply to all biofilms, independent of the causative organism producing the biofilm structure.

Pseudomonas aeruginosa, which is a gram-negative rod, is one of many organisms found in slime residues associated with a wide variety of industrial, commercial and processing operations such as sewerage discharges, re-circulating water systems (cooling tower, air conditioning systems etc.), water condensate collections, paper pulping operations and, in general, any water bearing, handling, processing, collection etc. systems. Just as biofilms are ubiquitous in water handling systems, it is not surprising that Pseudomonas aeruginosa is also found in association with these biofilms. In many cases, Pseudomonas aeruginosa is the major microbial component.

In addition to its importance in industrial processes, Pseudomonas aeruginosa and its associated biofilm structure have far-reaching medical implications, being the basis of many pathological conditions. Pseudomonas aeruginosa is an opportunistic bacterium that is associated with a wide variety of infections. It has the ability to grow at temperatures higher than many other bacteria and it is readily transferred from an environmental setting to become host-dependent. Translocation, both within a specific medium and to other media, is facilitated with its single polar flagella.

Pseudomonas aeruginosa has nutritional versatility in being able to use a wide variety of substrates, fast growth rate, motility, temperature resiliency and short incubation periods all of which contribute to it predominance in natural microflora communities as well as being the cause of nosocomial (hospital acquired) infections.

Infections caused by Pseudomonas aeruginosa begin usually with bacterial attachment to and colonization of mucosal and cutaneous tissues. The infection can proceed via extension to surrounding structures or infection may lead to bloodstream invasion, dissemination and sepsis syndrome. Virtually all infections that are caused by Pseudomonas aeruginosa as the principal cause, or as a confounding factor to other bacterial-based infections, are treatable. The one notable exception is cystic fibrosis where the infection is chronic.

Respiratory Infections. Alginate producing strains of Pseudomonas aeruginosa infect the lower respiratory tract of patients with cystic fibrosis leading to acute and the chronic progression of the pathological condition. In addition to cystic fibrosis, primary pneumonia often presents bilateral bronchopneumonia with nodular infiltrates. Accompanying such infections are pleural effusions along with pathological progression leading to alveolar necrosis, focal hemorrhages and micro-abscesses.

Mucoid strains Pseudomonas aeruginosa typically infect the lower respiratory tract of individuals with cystic fibrosis. Airway obstruction typically begins with bronchial infection and mucus production followed by colonization of Pseudomonas aeruginosa in the lower respiratory tract. The colonization of Pseudomonas aeruginosa accelerates disease pathology resulting in increased mucus production, airway obstruction, bronchiectasis and fibrosis in the lungs. These conditions eventually lead to pulmonary disease leading to hypertension and hypoxemia.

It is reasonable to expect a reduction in viscosity of the mucus if the biofilm produced by the bacteria were dismantled. Since the biofilm, can and likely does to a large measure, adhere to lung tissue surfaces, an enzyme that dismantles the biofilm, being equipped with an anchor would be effective in treating cystic fibrosis patients. The treatment with the enzyme-anchor complex would accomplish several key objectives for an effective treatment regime: 1) viscosity reduction of the sputum; 2) bacterial colony size reduction; and 3) bacteria exposition by reducing the biofilm so that antibiotics and the host's own immune system would be more effective.

Equipping DNase with an anchor and combining it with and enzyme-anchor complex having an enzyme that degrades the biofilm would allow greater retention of these two, though functionally different enzymes, for the treatment of cystic fibrosis.

While cystic fibrosis is a chronic infection of Pseudomonas aeruginosa, other, acute, respiratory infections occur as a result of bacteria other than Pseudomonas aeruginosa. For example, Streptococcus pyrogenes is the primary cause of bacterial pharyngitis which, is uncontrolled, can lead to rheumatic fever. Nelson, et al. [Proc. Acad. Sci. 98, 4107-4112(2001)] report a lysis process to control the bacterial infection using double-stranded DNA bacteriophages. The enzymes associated with the bacteriophage-mediated lysis serve as examples of implementing the present invention.

Alginate Biofilms of Pseudomonas aeruginosa: At the root of Pseudomonas aeruginosa initial colonization, as well as its proliferative growth rate, is the production of a mucoid exopolysaccharide layer comprised of alginate. This exopolysaccharide layer, along with lipopolysaccharide, protects the organism from direct antibody and complement mediated bactericidal mechanisms and from opsonophagocytosis. This protective biofilm allows Pseudomonas aeruginosa to expand, grow and to exist in harsh environments that may exist outside the alginate biofilm. It is not surprising that the alginate biofilm is considered as an important virulence factor.

The alginate biofilm or “slime matrix” consists of a secreted polysaccharide that serves as the backbone structure of the biofilm. Alginate is a polysaccharide copolymer of β-D-mannuronic acid and α-L-guluronic acid linked together by 1-4 linkages. The immediate precursor to the biosynthetic polymerization is guanosine 5′-diphosphate-mannuronic acid, which is converted to mannuronan. Post-polymerization of the mannuronan by acetylation at O-2 and O-3 and epimerization, principally at C-5, of some of the monomeric units to produce gulonate, results in varying degrees of acetylation and gulonate residues. Both the degree of acetylation and the percentage of mannuronic residues that have been converted to gulonate residues greatly affect the properties of the biofilm. For example, polymers rich in gulonate residues and in the presence of calcium, tend to be more rigid and stiff than polymers with low levels of gulonate monomeric units.

Construction of Anchor-Enzyme Complexes.

The Anchor Enzyme Complex can be constructed using chemical synthetic techniques. Additionally, the Anchor-Enzyme Complex, if the anchor is a polypeptide or protein, such as protein binding domains, lectins, selectins, heparin binding domains etc., can be constructed using recombinant genetic engineering techniques.

Types of Anchors.

The binding domain from elastase
Domains that bind to carbohydrates and polysaccharide
Lectins
Mannose Binding Lectin
Selectins
The binding domains from Heparin
The binding domains of Fibronectin
CD44 Protein
Additional anchors listed in U.S. Pat. No. 5,871,714.
   Type of Enzymes

1. Generally, enzymes in the class EC 4.2.2._, which are polysaccharide lyases, which degrade the polysaccharide backbone structure of biofilms:

Glycoside Hydrolases, Galactoaminidases, Galactosidases, Glucosaminidases, Glucosidases, Mannosidases (EC 3.1.2.-)

Neuraminidase (EC 3.1.2.18)

Dextranase, Mutanase, Mucinase, Amylase, Fructanase, Galactosidase, Muramidase, Levanase, Neuraminidase(EC 3.2.-)

a-Glucosidases (EC 3.2.1.20)

β-Glucosidase (EC 3.2.1.21)

α-Glucosidase (EC 3.2.1.22)

β-D-Mannosidase (EC 3.2.1.25)

Acetylglucosaminidase (EC 3.2.1.30)

Hyaluronoglucosaminidase (EC 3.2.1.35)

α-L-Fucosidase (EC 3.2.1.51)

Hyaluronate Lyase (EC 4.2.2.1)

Pectate Lyase (EC 4.2.2.2)

Alginate Lyase [Poly(β-D-Mannuronate) Lyase] (EC 4.2.2.3)

Chondroitin ABC Lyase (EC 4.2.2.4)

Chondroitin AC Lyase (EC 4.2.2.5)

Oligogalacturonide Lyase (EC 4.2.2.6)

Heparin Lyase (EC 4.2.2.7)

Heparan Lyase [Heparitin-Sulfate Lyase] (EC 4.2.2.8)

Exopolygalacturonate Lyase (EC 4.2.2.9)

Pectin Lyase (EC 4.2.2.10)

Poly (α-L-Guluronate) Lyase (EC 4.2.2.11)

Xanthan Lyase (EC 4.2.2.12)

Exo-(1,4)-α-D-Glucan Lyase (EC 4.2.2.13)

Non-specific polysaccharide depolymerases derived from bacteriophages et al.

2. Enzymes for removing debris either embedded within the biofilm structure or extraneous byproducts of both the biofilm and host-originated materials. This later debris may originate from the host and would include immune response products. These include many EC sub-classes with the general class of hydrolytic and digestive enzymes. In descriptive terms, they include enzymes that facilitate the breaking of chemical bonds and include the following:
Esterases—cleavage of ester bonds;
Glycolytic—cleavage of bonds found in oligo- and polysaccharides
Peptidases—cleavage of peptide bonds where the substrate is a protein or polypeptide;
Nucleic acid materials (RNA and DNA);
Carbon-nitrogen cleavage—where the substrate is not a protein or polypeptide;
Acid anhydride cleaving enzymes;
Carbon-carbon bond cleavage;
Halide bond cleavage;
Phosphorus-nitrogen bond cleavage;
Sulfur-nitrogen bond cleavage; and
Carbon-phosphorus bond cleavage.

Typical examples include the following enzymes:
Endopeptidases; Peptide Hydrolases (EC 3.4.-)
Aminopeptidases (EC 3.4.11)
Nucleic Acid Hydrolases (EC 3.1.-.-)
Propyl Aminopeptidases (EC 3.4.11.5)
Glycylpropyl Dipeptidases; Dipeptidyl Peptidase (EC 3.4.14)
Serine Endopeptidases (EC 3.4.21)
Chymotrypsin (EC 3.4.21.1)
Trypsin (EC 3.4.21.4)
Amidohydrolases (EC 3.5.-)
N-Acetylglucosamine-6-phosphate Deacetylase (EC 3.5.1.25)
Oxo-Acid Lyases (EC 4.1.3.-)
N-Acetylmuraminate Lyases (EC 4.1.3.3)
Carbohydrate Epimerases (EC 5.1.3-)
Glucosamine-6-phosphate Isomerases (EC 5.3.1.10)

### EXAMPLE

Since Pseudomonas aeruginosa is a ubiquitous bacterial strain, found not only in the environment and in industrial settings where fouling occurs, but also in many disease conditions, it will serve as an example to illustrate the principles of the invention. Further, while there are many disease conditions for which Pseudomonas aeruginosa is the cause, ocular infections will exemplify the implementation of the invention. The choice of Pseudomonas aeruginosa as the biofilm-producing bacteria and pathogen and ocular infection as a consequence of the biofilm is not meant to preclude or limit the scope of this invention. The principles outlined in this example readily apply to all biofilms, whether produced by bacteria or other organisms, all biofilms that are generated by organisms and the embodiments, taken and implemented either individually or collectively.

Pseudomonas aeruginosa is an opportunistic bacterial species, which once colonized at a site such as ocular tissue, produces a biofilm with a polysaccharide-based alginate polymer. This exopolysaccharide or glycocalyx matrix is the confine in which the bacterial species can grow and proliferate. This biofilm matrix can also serve as a medium for other, pathogenic bacteria, fungi and viruses. It is of therapeutic benefit, therefore, to remove the biofilm structure and eliminate all pathogens at the site, not only Pseudomonas aeruginosa.

Alginate lyase, the expression product from the algL gene, can be obtained from various bacterial sources e.g. Azotobacter vinelandii, Pseudomonas syringe, Pseudomonas aeruginosa etc., producing an enzyme AlgL, which degrades alginate. Other genes, e.g. alxM, also provide a wide variety of alginate lyase and polysaccharide depolymerase enzymes with degrade alginate by various mechanisms.

Endogenous lectins, heparin binding domains and various receptors from animals and plants have receptors that bind to alginate. These receptors, when located on host cell surfaces, allow the evolving alginate biofilm to be retained by the infected tissue. Elastase (Leukocyte Elastase, EC 3.4.21.37 and Pancreatic Elastase, EC 3.4.21.36), which is a digestive enzyme, also has a domain that binds to alginate. Such binding capability, along with the degradative ability of the catalytic site in elastase, has been implicated in tissue degradation associated with alginate biofilm infections such as cystic fibrosis. In addition, other serine proteases also have alginate binding domains.

In one aspect of the invention, a fusion protein is created, using standard genetic engineering techniques. One of the traits or elements of the fusion protein is the ability to degrade alginate and a second property being a binding capability of the newly-created fusion protein, derived from, for example, the binding domain of elastase. The bi-functional protein fulfills the criteria set out in the-invention in that the binding domain derived from elastase serves as the anchor and the alginate lyase portion of the fusion protein serves as the degradative enzyme for the biofilm.

It is within the scope of this invention that the principles outlined here also apply to all biofilms in all circumstances in which they occur.

Construction of the Enzyme Anchor Complex

Using molecular biology and biotechnology techniques, gene fusions are created to produce unique proteins from recombinant DNA segments. A DNA sequence, which specifically codes for an enzyme is fused to a DNA segment that specifically codes for a protein-binding domain. The resulting fused DNA segment will produce a unique protein that possesses both enzymatic or catalytic activity and binding activity.

The DNA sequence that codes for alginate lyase obtained from Pseudomonas aeruginosa, or another acceptable strain, was isolated and amplified using polymerase chain reaction. The sequence was subcloned into an expression vector. Next the DNA that codes for leukocyte elastase was isolated from a mouse complimentary DNA (cDNA) library. The mouse leukocyte elastase sequence was amplified by using polymerase chain reaction.

Both DNA sequences for alginate lyase and mouse leukocyte elastase were subcloned into a single open reading frame within a suitable expression vector. Thus, yielding a DNA sequence that codes for a single protein that contains both the amino acid sequence for alginate lyase as well as the sequence for leukocyte elastase. This hybrid or chimeric protein has the catalytic ability to degrade alginate as well as the binding ability of elastase.

Assay Procedure for Synthesized Anchor Enzyme Complexes

Preparation of Bacterial Biofilms. There are many procedures to prepare bacterial biofilms. Herein are two of those procedures.

Analysis of a Dynamic System. The appropriate bacterial strain, or mixed strains if more than one strain is used, is incubated in tryptic soy broth for 18 to 24 hours at 37° C. After the incubation period, the cells are washed three times with isotonic saline and re-suspended in isotonic saline to a density of 106 CFU/ml. The re-suspended cells are incubated a second time with Teflon squares (1×1 cm) with a thickness of 0.3 cm for six to seven days at 37° C. The recovered cells in the saline incubation medium are planktonic bacteria, while those associated with the Teflon squares and the biofilm are sessile cells.

The biofilm-associated sessile cells are then treated with appropriate anchor-enzyme complexes that degrade the generated biofilm at various concentrations with or without bactericidal agents in either a completely closed system or an open system (flow-through chamber or cell). The bactericidal agent can be either an anchor enzyme system that generates active oxygen or a non-enzymatic, chemical that is a recognized antimicrobial agent, biocide or antibiotic.

Analysis of a Static System. The Teflon squares with the associated biofilm are transferred to isotonic saline medium containing a given concentration of anchor-enzyme complex that degrades the biofilm. At intervals of 3, 6, 12, 24 and 48 hours, the individual Teflon squares are washed three times with isotonic saline and finally added to fresh isotonic saline which is vigorously shaken or sonicated for tow minutes. The suspended mixture is diluted and counted for cell density and expressed as number of CFU/ml.

The same counting procedure can be used for the incubation medium.

Bactericidal agents are also incorporated into the experimental design, which also uses the same cell counting procedure.

Estimating Biofilm Size. At the end of any of the incubation steps, the biofilm can be recovered, dehydrated and weighed to obtain total biomass of the biofilm. Alternatively, the amount of alginate backbone can be determined where the biofilm contains Pseudomonas sp.

Extraction of Polysaccharide Backbone. After the second incubation and disruption of the biofilm, the bacterial cells are removed from the dispersion. With an increasing concentration of an ethanol/soling gradient, the alginate is precipitated, collected and washed three times with 95% ethanol. The precipitate is desiccated after which the quantity can be determined gravimetrically or by any number of chemical, enzymatic or combination of chemical and enzymatic methods. The most widely used method is the chemical method of which there are three types: uronic acid assay, orcinol-FeCl3 and decarboxylation and CO2 measurement.

Analysis in a Dynamic System (Complete or Partial). The most widely used dynamic flow system that can be regulated from a completely closed to a completely open system is the Robbins Device or the Modified Robbins Device. The Modified Robbins Device allows the assessment of biofilms in which the fluid flow and growth rates of the biofilm can be regulated independently and simultaneously. A Robbins-type flow cell can be a completely closed system that possesses flow dynamics for assessing efficacy of anchor-enzyme complexes.

1. A composition for degrading biofilm structure associated with cystic fibrosis and the debris associated therewith, the composition comprising:
an enzyme selected for its ability to dismantle the biofilm structure;
an anchor molecule coupled to an enzyme to form an enzyme-anchor complex, the anchor molecule being selected for its ability to attach to a surface on or proximal the biofilm structure;
wherein the attachment to the surface permits prolonged retention time of the enzyme-anchor complex where the biofilm structure and associated debris are present.

2. A composition as claimed in claim 1 wherein the enzyme is selected for its ability to degrade a colonizing matrix.

3. A composition as claimed in claim 1 wherein the enzyme-anchor complex is a fusion protein.

4. A composition as claimed in claim 1 wherein the enzyme-anchor complex is constructed using chemical synthesis techniques.

5. A composition as claimed in claim 1 wherein the enzyme-anchor complex contains alginate lyase to degrade the biofilm structure.

6. A composition as claimed in claim 1 wherein the enzyme-anchor complex further contains DNase to degrade debris which are byproducts of the degraded biofilm structure.

7. A composition as claimed in claim 1 wherein the enzyme-anchor complex comprises an anchor having an alginate-binding domain.

8. A composition as claimed in claim 7 wherein the alginate-binding domain is derived from elastase.

9. A composition as claimed in claim 7 wherein the alginate-binding domain is derived from a glycosyltransferase enzyme.

10. A composition as claimed in claim 7 wherein the alginate-binding domain is derived from an alginate polymerase enzyme.

11. A composition as claimed in claim 7 wherein the alginate binding domain is a mannose binding lectin.

12. A composition as claimed in claim 7 wherein the alginate-binding domain is derived from heparin.

13. A composition as claimed in claim 7 wherein the alginate-binding domain is derived from fibronectin.

14. A composition as claimed in claim 7 wherein the alginate-binding domain is derived from Concanavalin A.

15. A composition as claimed in claim 7 wherein the alginate-binding domain is derived from a lectin.

16. A composition as claimed in claim 7 wherein the alginate-binding domain is derived from a selectin.

17. A composition as claimed in claim 7 wherein the alginate-binding domain is derived from the CD44 protein.

18. A composition as claimed in claim 1 further comprising an additional enzyme-anchor complex comprised of an enzyme selected for its ability to act upon debris and byproducts associated with the biofilm structure degradation coupled to an anchor selected for its ability to attach to a surface on or proximal the biofilm structure.

19. A composition claimed in claim 18 wherein the enzyme-anchor complex is a fusion protein.

20. A composition as claimed in claim 18 wherein the enzyme-anchor complex is constructed using chemical synthesis techniques.

21. A composition claimed in claim 18 wherein the additional enzyme-anchor complex comprises an anchor having an alginate-binding domain.

22. A composition claimed in claim 18 wherein the enzyme-anchor complex contains a proteinase.

23. A composition as claimed in claim 18 wherein the enzyme-anchor complex has the capability to act on DNA.

24. A composition claimed in claim 23 wherein the enzyme-anchor complex contains DNase.

25. A composition claimed in claim 18 wherein the enzyme-anchor complex contains mucinase.

26. A composition claimed in claim 18 wherein the enzyme-anchor complex is a cell wall degrading enzyme.

27. A composition claimed in claim 18 wherein the enzyme-anchor complex contains a glycosaminoglycan hydrolase.

28. A composition claimed in claim 18 wherein the enzyme-anchor complex contains a peptidoglycan hydrolase.

29. A composition claimed in claim 18 wherein the enzyme-anchor complex contains proteoglycan hydrolase.
